(19) United States
(12) Patent Application Publication
(10) Pub. No.: US 2010/0009355 A1
(43) Pub. Date: Jan. 14, 2010

(54) SELECTIVE AMPLIFICATION OF MINORITY MUTATIONS USING PRIMER BLOCKING HIGH-AFFINITY OLIGONUCLEOTIDES

(76) Inventor: Michael S. Kolodney, Playa Vista, CA (US)

Correspondence Address: Weaver Austin Villeneuve & Sampson LLP, P.O. BOX 70250, OAKLAND, CA, 94612-0250, US

(21) Appl. No.: 12/282,504

(22) PCT Filed: Mar. 14, 2007
(86) PCT No.: PCT/US07/06442

§ 371 Date: Jan. 12, 2009

Related U.S. Application Data
(60) Provisional application No. 60/782,711, filed on Mar. 14, 2006.

(51) Int. Cl. C12Q 1/68; C12P 19/34

(52) U.S. Cl. 435/6; 435/91.2

(57) ABSTRACT

In certain embodiments this invention pertains to methods of detecting and/or quantifying rare mutant nucleic acids in populations of nucleic acids in which the wild-type nucleic acids are in substantially greater abundance than the rare mutants. In various embodiments the methods utilize short high affinity oligonucleotides targeted to the wild type rather than the minority or mutant sequence. Rather than directly detecting mutant DNA, these probes block detection of wild type DNA. These “blocker” probes can be used in combination with longer “detection” probes or PCR primers to amplify and/or identify the minority mutation in, e.g., clinical specimens. The combination of short high affinity blocker probes and longer, lower affinity detection probes eliminates the single base specificity/complexity tradeoff in the design of nucleic acid probes.

### CROSS-REFERENCE TO RELATED APPLICATIONS

This application claims benefit of and priority to U.S. Ser. No. 60/782,711, filed Mar. 14, 2006, which is incorporated herein by reference in its entirety for all purposes.

### STATEMENT AS TO RIGHTS TO INVENTIONS MADE UNDER FEDERALLY SPONSORED RESEARCH AND DEVELOPMENT

Not Applicable

### FIELD OF THE INVENTION

This invention pertains to the field of nucleic acid detection. In particular this invention relates to the use of high affinity probes as blocking reagents to facilitate the detection of rare mutants in complex populations of nucleic acids.

### BACKGROUND OF THE INVENTION

Detection of single base mutations in heterogeneous specimens may improve cancer detection and aid in the targeting of mutation directed therapeutic agents. Allele Specific Polymerase Chain Reaction (AS-PCR) and Ligase Chain Reaction (LCR) based methods can detect a small amount of mutated DNA in the presence of excess wild type DNA (minority mutations). These techniques exploit the decreased efficiency of DNA polymerases and ligases in the presence of a single base mismatch at the 3′ terminus of an oligonucleotide. Thermostable polymerases and ligases, however, will inadvertently extend or ligate oligonucleotides with a 3′ terminal mismatch at a frequency of about one percent or greater (Ayyadevera et al. (2000) Anal Biochem., 284(1): 11-18; Baramy (1991) Proc. Natl. Acad. Sci., USA, 88: 89). Because these errors are propagated in subsequent cycles of the chain reaction, these methods can only detect mutations present at a frequency of about one percent or greater.

An alternative method to detect mutations uses labeled probes that selectively bind the mutant over the wild-type sequence. Several types of mutation specific probes have been developed including TAQMAN® probes, molecular beacons, and scorpions (Afonina et al. (2002) PharmaGenomics 48-54; Wong and Medrano (2005) Bio Techniques 39: 75-85). The single base selectivity of these probes can be improved by using high affinity nucleotide analogues such as peptide nucleic acid (PNA), locked nucleic acid (LNA) (Ugozzoli et al. (2004) Anal Biochem. 1: 143-152; Demidov (2003) Biotechnology 21: 4-7), or minor groove binding probes. These chemistries allow construction of short nucleic acid probes that bind with similar affinities to much longer natural DNA probes (Dominguez and Kolodney (2005) Oncogene, 24: 6830-6834). A desirable property of these short probes is the large decrease in binding affinity when one of the bases near the center of the probe is mismatched with its target sequence. This single base discrimination ability would make these short probes ideal for identifying single base mutations or polymorphisms. However, these short oligonucleotides lack complexity and therefore tend to bind to genomic DNA sequences other than the target because a short sequence may not be unique among the samples being tested.

### SUMMARY OF THE INVENTION

This invention pertains to methods of detecting and/or quantifying rare mutant nucleic acids in populations of nucleic acids in which the wild-type nucleic acids are in substantially greater abundance than the rare mutants. In various embodiments the methods utilize short high affinity oligonucleotides targeted to the wild type rather than the minority or mutant sequence. Rather than directly detecting mutant DNA, these probes block detection of wild type DNA. These “blocker” probes can be used in combination with longer “detection” probes or PCR primers to amplify and/or identify the minority mutation in, e.g., clinical specimens. The combination of short high affinity blocker probes and longer, lower affinity detection probes eliminates the single base specificity/complexity tradeoff in the design of nucleic acid probes.

Thus, in certain embodiments, this invention provides methods of preferentially amplifying a rare mutant nucleic acid in a population of nucleic acids comprising wild-type nucleic acids substantially in excess of the rare mutant nucleic acid. The methods typically involve carrying out a polymerase chain reaction (PCR) using a first primer and a second primer, where the first primer hybridizes with the region of the rare mutant nucleic acid comprising a mutation and the first primer and the second primer are not high affinity nucleic acids; where the reaction mixture of the polymerase chain reaction also contains a high affinity nucleic acid analog, the high affinity nucleic acid analog being complementary to the region of a wild-type nucleic acid that is mutated in the mutant nucleic acid; whereby binding of the high affinity nucleic acid analog to the wild-type nucleic acid prevents or reduces/inhibits the first primer from binding to the wild-type nucleic acid thereby resulting in the preferential amplification of the rare mutant nucleic acid. In certain embodiments these methods further involve recovering the amplification product produced by the polymerase chain reaction; diluting the amplification product; carrying out the polymerase chain reaction again with the first primer, the second primer, and the high affinity nucleic acid analogue to further preferentially amplify the rare mutant nucleic acid. In certain embodiments population at frequency of less than about 1 in 10³, or less than about 1 in 10⁴, or less than about 1 in 10⁶, or less than about 1 in 10⁸, or 1 in 10¹⁰. In certain embodiments the high affinity nucleic acid analogue is a locked nucleic acid (LNA), a peptide nucleic acid (PNA), a hexitol nucleic acid (HNA), a phosphoramidate, or other high-affinity nucleic acid. In certain embodiments the first primer and the second primer independently range in length from about 12 nucleotides to about 60 nucleotides, and/or independently range in length from about 8 nucleotides to about 30 nucleotides, and/or independently range in length from about 15 nucleotides to about 40 nucleotides. In certain embodiments the first primer is a forward primer. In certain embodiments the second primer is a forward primer. In various embodiments the high affinity nucleic acid analogue ranges in length from about 3 to about 25 bases, and/or from about 5 to about 15 bases, and/or from about 5 to about 10 bases. In certain embodiments, the high affinity nucleic acid analogue is present at a concentration of at least about 4-fold, at least about 8-fold, or at least about 10-fold, or at least about 15-fold or 20-fold greater than the concentration of the first primer. In certain embodiments the high affinity nucleic acid analogue is present at a concentration of at least about 10-fold, or at least about 15-fold or 20-fold greater than the concentration of the first primer. In certain embodiments the mutant nucleic acid comprises a one or a plurality of point mutations.

In certain embodiments this invention provides methods of detecting and/or quantifying a rare mutant nucleic acid in a population of nucleic acids comprising wild-type nucleic acids substantially in excess of the rare mutant nucleic acid. These methods typically involve hybridizing the rare mutant nucleic acid with a nucleic acid probe while blocking or reducing binding of the nucleic acid probe to the corresponding wild-type sequences by hybridizing the wild-type sequences to a high affinity nucleic acid analogue; and detecting the hybridized nucleic acid probe or performing one or more PCR amplification reactions and detecting the amplification product comprising the mutant nucleic acid. In certain embodiments the nucleic acid probe is labeled with a detectable label (e.g., a radioactive label, a radio-opaque label, an enzymatic label, a calorimetric label, a fluorescent label, and the like). In certain embodiments population at frequency of less than about 1 in 10², or less than about 1 in 10³, or less than about 1 in 10⁴, or less than about 1 in 10⁵. In certain embodiments the high affinity nucleic acid analogue is a locked nucleic acid (LNA), a peptide nucleic acid (PNA), a hexitol nucleic acid (HNA), a phosphoramidate, or other high-affinity nucleic acid. In various embodiments the nucleic acid probe ranges in length from about 12 nucleotides to about 100 nucleotides and/or from about 15 nucleotides to about 40 nucleotides, and/or from about 8 nucleotides to about 30 nucleotides. In various embodiments the high affinity nucleic acid analogue ranges in length from about 3 to about 25 bases, and/or from about 5 to about 15 bases, and/or from about 5 to about 10 bases. In certain embodiments, the high affinity nucleic acid analogue is present at a concentration of at least about 4-fold, at least about 8-fold, or at least about 10-fold, or at least about 15-fold or 20-fold greater than the concentration of the first primer. In various embodiments the mutant nucleic acid comprises one or a plurality of point mutations.

Also provided are methods of detecting and/or quantifying a rare mutant nucleic acid in a population of nucleic acids comprising wild-type nucleic acids substantially in excess of the rare mutant nucleic acid, where the methods involve carrying out a polymerase chain reaction (PCR) using a first primer and a second primer, where the first primer hybridizes with the region of the rare mutant nucleic acid comprising a mutation and the first primer and the second primer are not high affinity nucleic acids; where the reaction mixture of the polymerase chain reaction also contains a high affinity nucleic acid analog, the high affinity nucleic acid analog being complementary to the region of a wild-type nucleic acid that is mutated in the mutant nucleic acid; whereby binding of the high affinity nucleic acid analog to the wild-type nucleic acid reduces or prevents the first primer from binding to the wild-type nucleic acid thereby resulting in the preferential amplification of the rare mutant nucleic acid.

Methods are also provided for performing a nucleic acid hybridization to a rare mutant nucleic acid in a population of nucleic acids comprising wild-type nucleic acids substantially in excess of the rare mutant nucleic acid, where the methods involve hybridizing the rare mutant nucleic acid with a nucleic acid probe or primer, while fully or partially blocking binding of the nucleic acid probe or primer to corresponding wild-type sequences by hybridizing the wild-type sequences to a high affinity nucleic acid analogue.

In certain embodiments methods are provided for detecting rare mutant nucleic acids in a complex population of nucleic acids, where the methods involve contacting the population of nucleic acids with a high affinity nucleic acid that specifically hybridizes with the region of the wild-type sequence in which the mutant is expected to occur; thereby blocking (partially or fully) the wild-type sequence; and contacting the population of nucleic acids with a probe to detect the wild-type sequence; or contacting the population of nucleic acids with a pair of PCR primers where one member of the pair hybridizes to a region of a nucleic acid in the population containing the mutation characterizing the rare mutants; and amplifying the rare mutant nucleic acid.

Methods are also provided for detecting a mutant nucleic acid in a mammal, where the methods involve providing a nucleic acid sample from the mammal; hybridizing the mutant nucleic acid with a nucleic acid probe or PCR primer, while blocking binding of the nucleic acid probe or primer to corresponding wild-type sequences by hybridizing the wild-type sequences to a high affinity nucleic acid analogue; and detecting the hybridized nucleic acid probe or performing one or more PCR amplification reactions and detecting the amplification product comprising the mutant nucleic acid. It will be appreciated that the “providing the nucleic acid step” need not be performed by the same person(s) performing the rest of the assay. Thus, for example, the sample can be provided by a clinician, while the assay is run in a laboratory.

Methods are also provided for screening an agent for the ability to induce or prevent a mutation in a nucleic acid. The methods typically involve contacting a cell comprising the nucleic acid with the test agent; providing a nucleic acid sample from the cell; performing one or more of the assays described herein to detect a rare/mutant nucleic acid where the presence or increase in frequency of the mutation (e.g. as compared to a control) is an indicator that the test agent induces the mutation and a decrease in mutation (e.g., as compared to a control) indicates the test agent reduces mutation. In various embodiments the control is the cell or animal exposed to no test agent or to the test agent at a lower concentration. The control can be from the same animal or cell at a different time for from similar animal(s) or cells. In various embodiments the test agent is administered to or contacted to a non-human mammal comprising the cell. In certain embodiments the test agent is added to a cell culture comprising the cell.

### DEFINITIONS

A “high-affinity nucleic acid analogue” refers to a modified nucleic acid that hybridizes to a complementary deoxyribonucleic acid target with higher affinity than a deoxyribonucleic acid probe having the same base sequence. High-affinity nucleic acids include, but are not limited to locked nucleic acids (LNAs), peptide nucleic acid (PNA), hexitol nucleic acids (HNAs), phosphoramidates, and the like.

A “Locked Nucleic Acid” (LNA) is a nucleic acid analogue (as polymer of purine and/or pyrimidine bases) characterized by the presence of one or more monomers athat are conformationally restricted nucleotide analogue with an extra 2h-O, 4h-C— methylene bridge added to the ribose ring. LNA has been defined as an oligonucleotide containing one or more 2h-O, 4h-C-methylene-(D-ribofuranosyl) nucleotide monomers. Such oligonucleotides that contain LNA monomers have shown stability towards 3h-exonucleolytic degradation and greatly enhanced thermal stability when hybridized to complementary DNA and RNA.

In the phrase “a rare mutant nucleic acid in a population of nucleic acids comprising wild-type nucleic acids” the wild-type nucleic acids refers to the predominant nucleic acid sequences while the “mutant” nucleic acids refers to a subset of nucleic acids that differ from the wild-type by changes in one or more (typically no more than a few) bases comprising the sequences. In certain embodiments the wild-type nucleic acids are present in at least 100-fold excess, more preferably in at least 1,000-fold, or 10,000-fold excess, and most preferably in at least 10⁶, 10⁷, or 10⁸-fold excess over the mutant nucleic acids.

The term “test agent” refers to an agent that is to be screened in one or more of the assays described herein for the detection of agents that induce mutation(s) or suppress mutations. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part 1, chapt 2, Overview of principles of hybridization and the strategy of nucleic acid probe assays, Elsevier, N.Y. (Tijssen). Generally, highly stringent hybridization and wash conditions are selected to be about 5 C lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42C using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72 C for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

### BRIEF DESCRIPTION OF THE DRAWINGS

FIG. 1 provides a schematic illustration of Primer Blocking PCR (PB-PCR). Wild-type and mutant genomic DNA are amplified in the presence of a short nigh affinity nucleic acid analogue (e.g., Locked Nucleic Acid (LNA)) oligonucleotide complimentary to a wild-type sequence that overlaps the forward primer binding site. In the presence of mutant template, a single-base mismatch between the short LNA blocking oligonucleotide and the mutant template prevents the blocker from annealing thereby allowing the forward primer to bind and amplify the mutant sequence. The LNA blocker binds with high affinity to its perfectly matched complementary sequence on the wild-type template. Since the LNA binding site overlaps with the primer binding site, the forward primer is unable to anneal and amplification of wild-type sequence is blocked.

FIGS. 2A and 2B illustrate real-time PCR amplification of wild type and mutant template in the presence or absence of blocker. Wild type or T1796A mutant genomic DNA was amplified in the absence and presence of LNA blocker. FIG. 2A: Amplification of 10⁴ copies of wild-type template in the absence (purple) or presence (blue) of LNA blocker. FIG. 2A: Amplification of 10⁴ copies of mutant template in the absence (blue) or presence (purple) of LNA blocker.

FIGS. 3A and 3B illustrate the specificity and sensitivity of primer-blocking PCR FIG. 3A: Samples containing increasing copies of wild-type or mutant template were amplified in the absence and presence of LNA blocker. For each sample, the cycle of amplification was measured as the Ct corresponding to a Delta Rn of 0.5. FIG. 3B: Real-Time PCR plot demonstrating the amplification of 10⁴ copies of wild-type template (grey) and amplification of 10 copies of mutant template (blue) in the presence of LNA blocker.

FIGS. 4A and 4B illustrate detection of BRAF TG1796-7AT tandem mutation. FIG. 4A: 10⁴ copies of TG 1796-7AT mutant genomic DNA was amplified in the absence (green) or presence (purple) of LNA blocker. FIG. 4B: Amplification of 10⁴ copies wild-type genomic DNA (red) or 10 copies of tandem mutated genomic DNA (green) in the presence of LNA blocker.

FIGS. 5A and 5B illustrate Amplification of mutant template mixed with an excess of wild-type. FIG. 5A: Wild-type genomic DNA from 10⁴ cells with (purple) or without (blue) the addition of BRAF T1796A point-mutated DNA from 10 cells was amplified in the presence of LNA blocker. FIG. 5B: Wild-type genomic DNA from 10⁴ cells with (grey) or without (blue) the addition of TG1796-7AT tandem mutated DNA from 10 cells was amplified in the presence of LNA blocker.

FIGS. 6A and 6B show selective amplification of 10⁵ copies of wild-type genomic DNA and sensitive amplification of 10 copies of BRAF mutated genomic DNA sing one-step real-time PCR, respectively. FIG. 6A; FIG. 2a. Real-time PCR amplification of samples containing 10⁵ copies of wild-type genomic DNA were analyzed. Samples underwent PCR for 99 cycles. Wild-type BRAF from genomic DNA was amplified with (i) non-AS primers and no LNA blocker, (ii) AS primers, and (iii) AS primers and LNA blocker. FIG. 6B: Real-time PCR amplification of samples containing 10 copies of V600E mutant BRAF genomic DNA. Samples underwent PCR for 99 cycles. Mutant BRAF from genomic DNA was amplified with (i) non-AS primers and no LNA blocker, (ii) AS primers, and (iii) AS primers and LNA blocker.

FIG. 7 shows elective Amplification of 10¹ copies of mutant BRAF using PBAS-PCR. Two-step PBAS-PCR (first step not shown) using genomic DNA isolated from mutant (A375M) and wild-type (HEK 293T) Cell Lines. 10 copies of mutant BRAF amplified at cycle 40, while amplification of wild-type BRAF was inhibited after 60 cycles.

FIG. 8 shows detection of circulating melanoma cells in whole human blood using PBAS-PCR. 1 mL of whole human blood was spiked with a defined number of BRAF mutated melanoma (A375M) cells ranging from 100 to 10⁴ cells/mL of blood. After cell enrichment by antibody-mediated negative selection, whole genome amplification was performed before PBAS-PCR to facilitate earlier amplification of all samples. Samples were amplified using two-step PBAS-PCR (first step not shown). The first step amplified DNA using non-AS primers and an LNA blocker for 30 cycles, and the second step used both AS-primers and an LNA blocker for 99 cycles. In both steps, amplification of mutant and wild-type BRAF was analyzed using real-time PCR to visualize the dose dependency of PBAS-PCR.

### DETAILED DESCRIPTION

The development of high affinity oligonucleotide analogue chemistries allows construction of short nucleic acid probes that bind with affinities similar to longer natural DNA probes. We have found that very short, high affinity nucleic acid analogues (e.g., oligonucleotide analogue probes (in some embodiments, 5-10 bases)) exhibit a large decrease in affinity for their targets when one of the bases near the center of the probe is mismatched. This single base discrimination ability would make these short probes ideal for identifying single base mutations or polymorphisms. However, these short oligonucleotide analogue probes tend to bind to sequences other than the target nucleic acid sequence because, unlike a longer sequence, a short sequence will not be unique among the sample being tested.

We have developed an approach to combine the mutation sensitivity of short, high affinity nucleic acid analogue oligonucleotides with the sequence specificity of longer natural DNA probes. This method circumvents the sensitivity/specificity tradeoff in the design of mutation specific nucleic acid probes. In our approach, one designs a short unlabeled “blocker oligonucleotide” using a high affinity nucleic acid analog such as locked nucleic acid (LNA), peptide nucleic acid (PNA), and the like. One then designs a longer, labeled probe (Detection Probe) made of normal (non-high affinity). Even though the detection probe is longer, its melting point is lower than the blocking probe because the detection probe is composed of lower affinity natural DNA rather than high affinity nucleic acid analogue.

In various embodiments, the blocking probe and detection probe are mixed with the target nucleic acid (template). The mixture is heated to separate the two DNA strands of the target nucleic acid sequence. As the target nucleic acid is cooled, the blocking probe binds to wild type DNA. The short blocker probe will not bind to mutant DNA because the single base difference causes a large difference in its binding affinity. As the DNA is cooled further, the detection probe binds to its complementary sequence. However, the detection probe does not bind to wild type DNA because the blocker oligonucleotide has bound to it first. Thus, the detection probe provides specificity for the correct sequence of DNA while the blocker probe provides sensitivity to a single base mutation. This blocker/detector approach can be used to provide sequence specificity with single base sensitivity in applications such as microarrays, real time PCR, fluorescent in situ hybridization (FISH), northern blotting or other mutation detection approaches.

The methods described herein can be performed with a number of kinds of high-affinity nucleic acid analogues. Such analogues are characterized by the ability to bind a nucleic acid template (e.g. a deoxyribonucleic acid) with an affinity greater than that shown by a deoxyribonucleic acid probe having the same base sequence (i.e., a sequence complementary to the template). High-affinity nucleic acid analogues are well known to those of skill in the art. Such analogues include, but are not limited to Locked Nucleic acids (LNA), peptide nucleic acids (PNAs) (see, e.g., Egholmet al. (1993) Nature (London), 365, 566-568; Hyrup and Nielsen (1996) Bioorg. Med. Chem. 4: 5-23, and the like), hexitol nucleic acids (HNAs) (see, e.g., Hendrix et al. (1997) Chem. Eur. J. 3: 110-120; Hendrixet aL (1997) Chem. Eur. J., 3: 1513-1520), phorphorarnidates (e.g. 2h-fluoro N3h-phosphoramidates (see, e.g., Schultzand Gryaznov (1996) Nucleic Acids Res. 24: 2966-2973, and the like)).

The high-affinity nucleic acid analogues are typically relatively short (e.g. from about 3 to about 25 bases, preferably from about 4 to about 20 bases, more preferably from about 5 to about 15 bases, and most preferably from about 5, 6, or 7 to about 9, 10, 11, 12, 13, or 14 bases). In certain embodiments the sequence of the nucleic acid analogues are selected so the analogues are complementary to the region of the wild-type nucleic acid in which it is desired to find one or more mutants. In various embodiments the nucleic acid analogue length is selected so that the analogue binds to the wild-type target, but not to nucleic acids comprising one or more mutations in the “target” sequence.

When the above-described method is utilized in polymerase chain reaction (PCR) assays the PCR reactions are carried out according to standard methods well known to those of skill in the art. The amplification template can be provided from any of a number of sources including, but not limited to isolated genomic DNA, reverse transcribed mRNA, CDNA, and the like.

The primers are selected according to standard methods to amplify the nucleic acid of interest. Typically at least one of the primers (e.g., the forward primer or the reverse primer) is selected to span the template region where the mutant(s) that are to be detected are expected to occur. In certain embodiments the primer(s) need not span the location of the mutation(s) but are simply close enough to the mutation(s) that in wild-type templates where the high affinity “blocker” binds the template, there is sufficient overlap between the high affinity blocker and the primer that proper annealing and/or extensions of the primer is prevented.

In various embodiments the primers range in length from about 6 or 8 or 10 nucleotides to about 80, 60, 40, 30, 25, or 20 nucleotides in length. In certain embodiments the primers range in length from about 8 or 10 or 12 nucleotides in length to about 15, 18, 20, or 25 nucleotides in length.

The PCR reaction is carried out according to standard methods well known to htsoe of skill in the art. PCR protocols are provided in detail, for example, Diffenbach and Dveksler, eds. (2003) PCR Primer: A Laboratory Manual (Cold Spring Harbor Laboratory Press; Innis et al. (1989) PCR Protocols: A Guide to Methods and Applications, Academic Press, Altshuler (2006) PCR Troubleshooting: The Essential Guide, Caister Academic Press, and the like. Illustrative protocols for the practice of the methods described herein are also illustrated herein in Examples 1 and 2. These examples are not intended to be limiting. Using the teaching provided herein, one of skill can readily apply the methods disclosed herein to other PCR reactions, and/or to other systems that utilize nucleic acid hybridization to detect rare species in a population of nucleic acids.

Moreover, as indicated above, the use of high-affinity “blocker” probes with longer lower affinity “detector” probes can find use in a number of contexts. These include, but are not limited to applications such as microarrays, real time PCR, fluorescent in situ hybridization (FISH), northern blotting or other mutation detection approaches.

The methods of this invention find use in a wide variety of contexts. For example, the methods can be used in forensic applications to characterize and identify specific genotypes, particularly genotypes comprising certain rare alleles or mutations. The methods find utility in pharmacogenomics to characterize particular phenotypes or pathologies expected to respond to certain medications. The methods can be used to quickly screen for and identify certain variants of pathogens (e.g., virus, bacteria, parasite, etc.), to characterize particular cancers, and the like.

In certain embodiments, the methods of this invention can be used to screen test agents for the ability to induce one nor more mutations or to suppress the formation of such mutations. The methods typically involve contacting a cell comprising the nucleic acid with the test agent; providing a nucleic acid sample from the cell; and screening that nucleic acid for the appearance of a rare mutation as described herein. A decrease in mutation, particularly when the cell or test animal is exposed to one or more mutagens or is a model for the formation of certain mutants, indicates that the test agent inhibits formation of mutations. Conversely, an increase of such mutations indicates that the test agent is a mutagen.

These applications are simply illustrative and not intended to limit the scope of the claimed invention. Using the teaching provided herein, other applications will be available to those of skill in the art.

In certain embodiments, this invention contemplates kits for performing one or more of the assays described herein. Typically such kits will include one or more detection probes (e.g., PCR primers or probes) and one or more high-affinnity nucleic acids that bind to the wild-region of the target molecule(s) as described herein.

The kits can optionally contain additional materials for the collection of blood, and/or the isolation of cells and/or DNA, and/or RNA, and the like.

In addition, the kits can, optionally, include instructional materials containing directions (i.e., protocols) for the practice of the methods of this invention. Preferred instructional materials provide protocols utilizing the kit contents for detecting the occurrence of rare nucleic acids in complex populations of nucleic acids, e.g., as described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Selective Amplification of Minority Mutations Using Primer Blocking LNA Substituted Oligonucleotides

In this example, we demonstrate the utility of short high affinity oligonucleotides targeted to the wild type rather than the minority or mutant sequence. Rather than directly detecting mutant DNA, these probes block detection of wild type DNA. These “blocker” probes can be used in combination with longer “detection” probes to identify minority mutation in clinical specimens. The combination of short high affinity blocker probes and longer, lower affinity detection probes eliminates the single base specificity/complexity tradeoff in the design of nucleic acid probes.

In our approach, a short unlabeled “blocker oligonucleotide” is designed by using a high affinity nucleic acid analog such as LNA or PNA. A longer, natural DNA, labeled probe (detection probe) or PCR primer is then synthesized. Although the detection probe is longer than the blocking probe, its melting point is lower because the detection probe is composed of lower affinity natural DNA rather than high affinity nucleic acid analogue. The blocking and detection probes are mixed with the target nucleic acid. As the mixture is cooled, the blocking probe will bind to wild-type DNA but not to mutant DNA because the single base mismatch between probe and mutant DNA causes a large difference in the binding affinity of the short probe. As the DNA is cooled further, the detection probe will bind to its complementary sequence. However, the detection probe will not bind to wild type DNA because the blocker oligonucleotide has bound to it first and is sterically blocking a portion of the longer probe's binding site. Thus, the detection probe provides the specificity to identify the target DNA sequence while the blocker probe provides sensitivity to a single base change. This blocker/detector approach could be applied to provide sequence specificity with single base sensitivity in applications such as microarrays, real time PCR, fluorescent in situ hybridization (FISH), northern blotting or other mutation detection approaches.

In this example, we demonstrate the feasibility of the blocker/detector approach in a real-time PCR assay that detects a point mutation in the BRAF gene. As illustrated in FIG. 1, a short LNA blocker binds the wild-type sequence at a location overlapping with the forward primer binding site. The blocker prevents binding of forward primer to wild-type template thereby preventing amplification of wild-type DNA. However, the blocker binds weakly to mutated sequences, allowing the forward primer to bind and amplify mutated DNA. Because inadvertently amplified wild-type sequences are blocked in subsequent cycles (until blocking capacity is overwhelmed), this approach can potentially detect a very small amount of mutant DNA in a large excess of wild-type DNA.

### Materials And Methods

Cell lines, Tissue Sources, and DNA Preparation

Human Genomic DNA was purchased from Roche Diagnostics (Indianapolis, 1N, USA). Mutant genomic DNA was isolated from cell lines using DNAzol® (Molecular Research Center, Inc., Cincinnati, Ohio, USA) as recommended by the manufacturer. Genomic BRAF T1796A DNA was extracted from the A375 cell line, which is homozygous for this mutation. Genomic TG1796-7AT DNA (Tandem mutation) was extracted from the WM266 cell line. Cell lines were obtained from ATCC (Manassas, Va., USA).

DNA and LNA Oligonucleotides

The forward primer sequence was based on the allele-specific primer described by Jarry et al. (2004) Molecular and Cellular Probes, 18: 349-352. We modified their primer by eliminating the 3′ terminus allele-specific base, creating a primer directly adjacent to the V600 mutation site. The reverse primer was designed to produce a 114 base pair product. The LNA blocker used was identical to that previously described by Dominguez and Kolodney (2005) Oncogene, 24: 6830-6834. All primers and LNA blockers were purchased from Integrated DNA Technologies (Coralville, Iowa, USA) with the following sequences:

Forward Primer:

(SEQ ID NO: 1)

5′- AGG TGA TTT TGG TCT AGC TAC AG-3′;
Reverse Primer:

(SEQ ID NO: 2)

5′- ATC AGT GGA AAA ATA GCC TCA ATT CT-3′;
and
LNA Blocker:

(SEQ ID NO: 3)

5′- GCT ACA GTG AGG G 3′.

PCR Parameters

The forward and reverse primer set was combined in Primer Mix A or Primer Mix B. Both reaction mixtures consisted of QuantiTect SYBR Green PCR Kit master mix purchased from Qiagen (Valencia, Calif., USA), 15 μM of each primer and either wild-type or mutant template. Additionally, 100 μM LNA blocker was added to Primer Mix B. The reaction was amplified in the ABI Prism 7000 mutation detection system (Foster City, Calif., USA). The cycling conditions used were one cycle at 95° C. for 15 minutes, 42 to 50 cycles at 95° C. for 15 seconds, 66° C. for 30 seconds, and elongation at 72° C. for 30 seconds, with fluorescence measured following elongation (Demidov (2003) Biotechnology 21: 4-7). A dissociation phase followed in which samples were subjected to a temperature ramp from 60 to 95° C.

To test the ability of the primer-blocking method to identify a small amount of a minority mutation in an excess of wild-type template, ten copies of a mutant template and 10⁴ copies of wild-type template were added to Primer Mix B.

### Results

Effect of Primer-Blocking LNA Oligonucleotide on Wild-Type and Mutant Templates

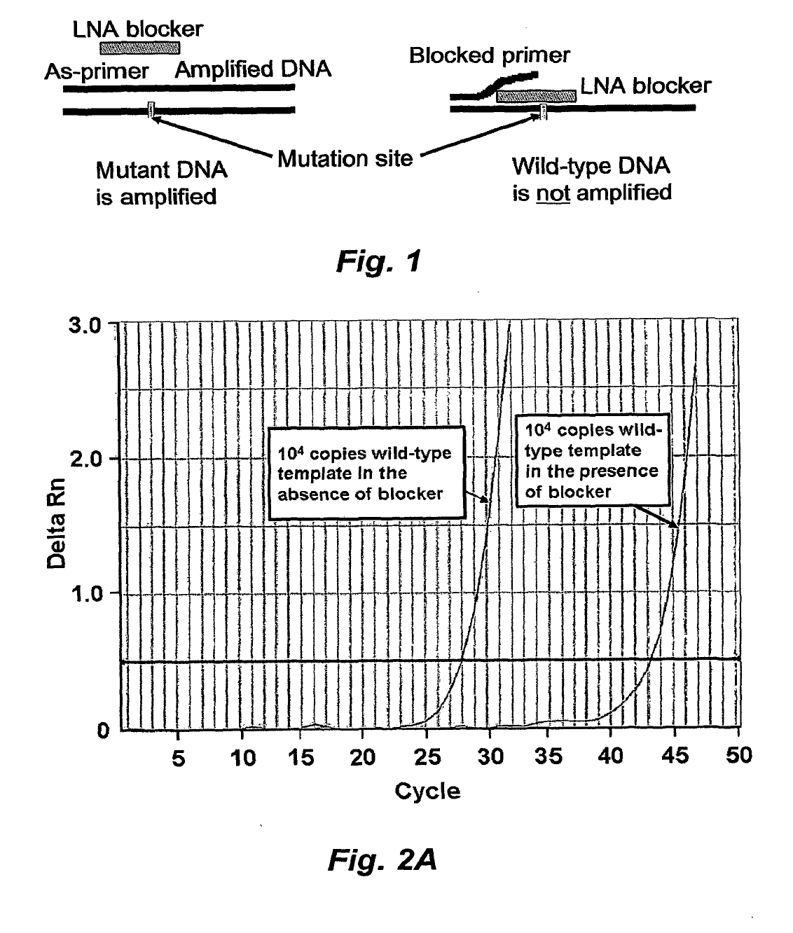

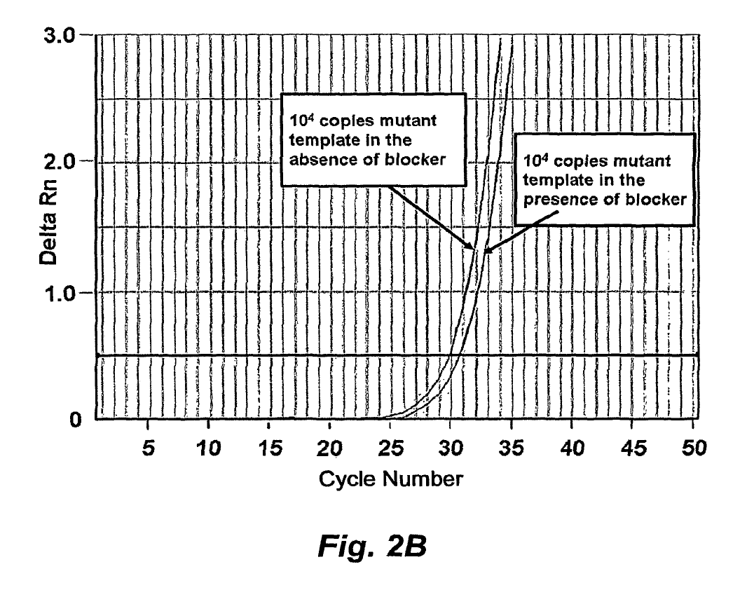

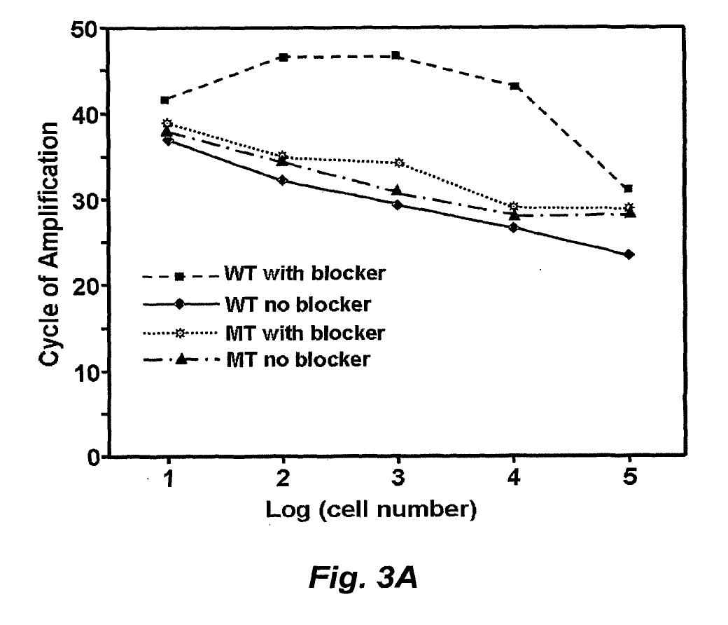

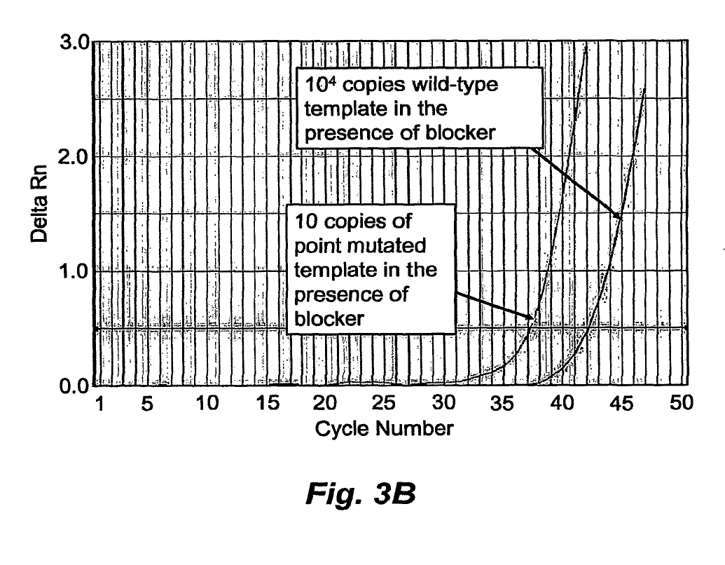

We tested the ability of a blocking LNA oligonucleotide to inhibit amplification of wild-type template by determining the delay caused by the blocker. Amplification of 10⁴ copies of wild-type BRAF template was delayed by fifteen cycles in the presence of 100 μM blocker (FIG. 2A). In contrast to its large inhibition of wild type amplification, the blocking oligonucleotide only delayed mutant amplification by one cycle (FIG. 2B). We determined the ability of primer-blocking PCR to identify minority mutations by determining the number of copies of wild-type BRAF that could be blocked under conditions that would amplify ten copies of T1796A mutated template. In this experiment, various concentrations of template were amplified in the presence of blocker. While all tested concentrations of wild-type template were greatly delayed by the LNA blocking oligonucleotide, mutant template was only slightly affected (FIG. 3A). Whereas ten copies of mutant template amplified at 38 cycles, 10⁴ copies of wild-type template did not amplify until the 42nd cycle (FIG. 3B). Thus the assay is able to detect ten copies of mutant template in a defined mixture containing a 10³ fold excess of wild-type template.

Amplification of TG1796-7AT Tandem Mutated Template

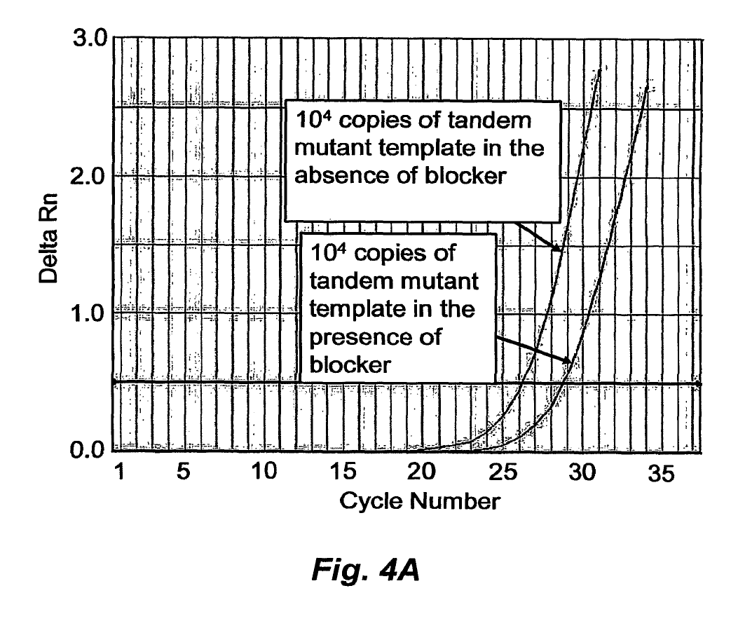

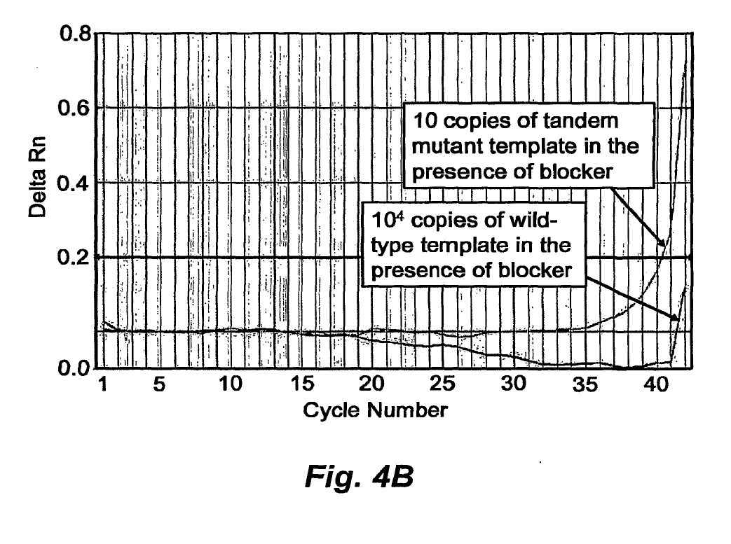

A major advantage of the primer-blocking method is its ability to identify multiple mutations in the region of the blocking oligonucleotide rather than being limited to a specific point mutation. To explore the potential of this method to identify more than one mutation using a single protocol, we tested the effect of the LNA blocker on TG1796-7AT tandem mutated template under the same conditions used to test the T1796A point mutated BRAF. Similar to point-mutated template, in the presence of blocker, amplification of 10⁴ copies of tandem mutated template was delayed by only 2.5 cycles (FIG. 4A). We also tested the utility of our assay to amplify ten copies of the tandem mutant under the same conditions that would amplify ten copies of Ti 796A mutated template and inhibit the amplification of a 10³ fold excess of wild-type. We found that ten copies of tandem mutated template amplified by 40 cycles while 10⁴ copies of wild-type template could not amplify until past the 42^{nd} cycle (FIG. 4B). Thus using a single protocol, our primer-blocking method can detect more than one mutation in the region of the blocking oligonucleotide.

Detection of Minority Mutations in a Defined Mixture of Genomic DNA

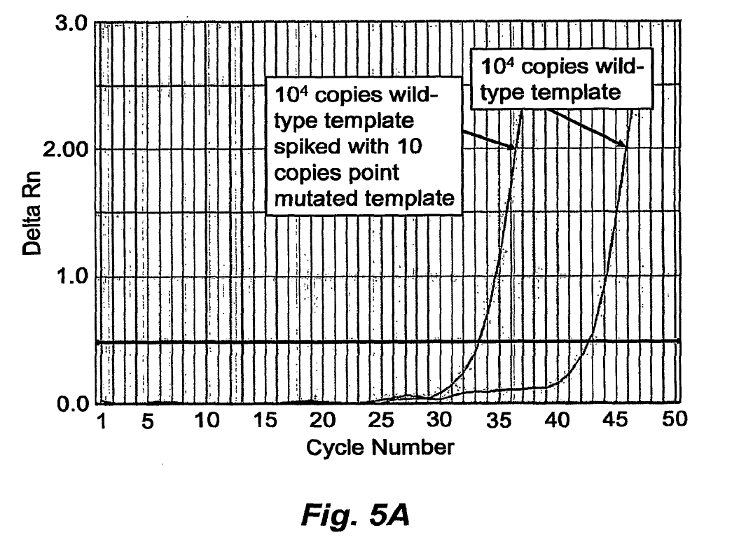

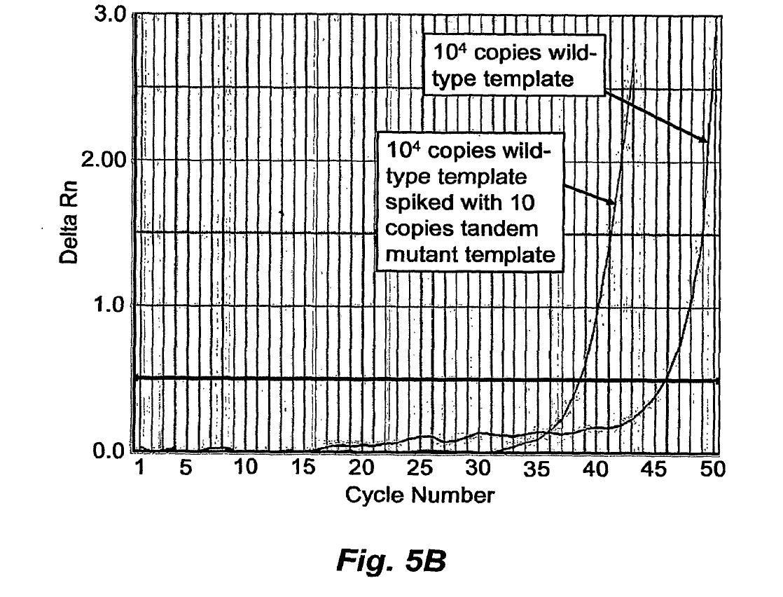

In order to test the utility of our primer-blocking PCR assay to detect minority mutations in a mixture of cell lines, we mixed wild-type genomic DNA with small amounts of genomic DNA from cell lines with mutant BRAF. In the first mixture, we added ten copies of the Ti 796A point-mutated template to 10⁴ copies of wild type template, in the presence of 100 uM LNA blocker. In a second mixture, we combined ten copies of TG1796-7AT tandem mutated DNA with 10⁴ copies of wild-type template. We compared the cycle of amplification of each of these mixtures with the cycle of amplification of a sample containing only 10⁴ copies of wild type template. As demonstrated in FIGS. 5A and 5B, the mixture of single-base mutated template plus wild-type template amplified at 33 cycles, while, the sample containing only wild-type template did not amplify until the 43^{rd} cycle (FIG. 5A). Similarly, the mixture containing ten copies of tandem mutant template amplified at 39 cycles whereas the sample containing wild type template alone did not amplify until the 46th cycle (FIG. 5B). Therefore, our primer-blocking method can be used to detect a minority mutation in the presence of a 10³ fold excess of wild-type DNA.

### Discussion

We have combined a novel blocker/detector approach with real-time PCR to identify single base minority mutations. There are several advantages to this new approach over conventional AS-PCR and LCR. At the molecular level, all allele specific amplification techniques fail a certain percentage of the time. AS-PCR and LCR will inadvertently produce mutant product from wild type template about 1% of the time (Ayyadevera et al. (2000) Anal Biochem., 284(1): 11-18; Baramy (1991) Proc. Natl. Acad. Sci., USA, 88: 89). These errors will be propagated in subsequent cycles thereby limiting the sensitivity of these techniques for low frequency mutations. In contrast, when the primer/blocker technique inadvertently amplifies a wild type template, a wild type product will be synthesized. Because this molecule has the wild-type sequence, its amplification will be blocked in future cycles. Another advantage of primer blocking PCR over conventional AS-PCR is that the primer/blocker assay is not restricted to a specific mutation. Since the assay functions by preventing the amplification of a wild-type sequence rather than enhancing amplification of a specific mutant sequence, it allows for the detection of any mutation located near the center of the blocker binding region. Other methods for detecting minority mutations use high affinity blocker sequences to prevent elongation of the amplicon rather than binding of the PCR primer. “PCR Clamping” uses high affinity PNA oligonucleotides as blockers (Orum et al. (1993) Nucleic Acid Res., 21: 5332-5336). Since PNA chemistry is resistant to Taq exonuclease activity, PNA oligonucleotides effectively block progression of the polymerase. However, PNA chemistry is poorly adaptable to current automated synthesizers greatly limiting the practicality of this approach. “Wild Type Blocking PCR” uses LNA oligonucleotides to block elongation of the amplicon. However, since LNA chemistry is susceptible to Taq exonuclease activity, the exonuclease deficient Stoffel fragment of Taq must be used (Dominguez and Kolodney (2005) Oncogene, 24: 6830-6834). The lack of a requirement for Stoffel fragment in primer blocking PCR allows the use of commercial real-time PCR master mixes and should also be compatible with hydrolysis based detection probes.

In summary, we have developed a novel blocker/detector approach, combining a short high affinity LNA blocker probe with a longer but lower affinity detection probe, to detect single base minority mutations. Advantages of the primer-blocking method include compatibility with real-time PCR, lack of error propagation in subsequent cycles, and an ability to detect minority mutations independent of polymerase fidelity. We expect that similar approaches using short, high affinity blocker probes combined with longer but lower affinity detection probes can be used to detect mutations in other nucleic acid hybridization methods such as FISH, “blotting” and micro-arrays.

### Example 2

### Detection of Rare Cancer Cells in Blood Using Primer-Blocking Allele-Specific PCR and Whole Genome Amplification

Detection of mutated genomic DNA from cancer cells circulating in blood may improve tumor staging and drug targeting. However, detecting a few mutated cells in a large (10⁶ fold) excess of wild-type cells requires a sensitive and selective assay. In this example, we describe a novel approach to detect circulating melanoma cells harboring a common mutation in the BRAF kinase. In the first step of our approach, a high affinity locked nucleic acid (LNA) oligonucleotide was used to block PCR amplification of wild-type BRAF while permitting amplification of mutant BRAF. In the second step, the LNA blocking approach was combined with a mutant-specific forward primer. This two-step approach easily detected ten BRAF mutated melanoma cells in the presence of 10⁵ wild-type cells. To determine the clinical utility of this method, we tested the ability of our method to detect human blood spiked with a defined number of BRAF mutated melanoma cells. Blood was first enriched for melanoma cells using an antibody-mediated negative selection procedure. Whole genome amplification (WGA) was performed on the enriched cells. WGA-amplified genomic DNA was then analyzed by two-step real-time PCR to detect the BRAF mutation. Using this approach, we could readily identify mutant DNA from as few as ten melanoma cells in 1 ml of human blood.

### Introduction

A substantial fraction of melanomas contain a point or tandem oncogenic mutation in exon 15 of BRAF, a cytoplasmic serine/threonine kinase in the MAPK pathway (Kumar et al. (2003) Clin. Cancer Res., 9: 3362-3368). These mutations cause constitutive activation of BRAF resulting in downstream activation of the MEK/ERK pathway (Davies et al. (2002) Nature 2002; 417: 949-954). Because of the high prevalence of oncogenic BRAF mutations in specific cancers, methods have been developed to detect these mutations in clinical specimens. Since most clinical specimens contain a mixture of cell types, these approaches must be able to detect mutations in tissue samples that often contain an excess of wild-type DNA mixed with mutant DNA.

Allele-specific PCR (AS-PCR) can detect one mutant copy of genomic DNA in 10² wild-type copies (Jarry et al. (2004) Molecular and Cellular Probes 18: 349-352). This method utilizes a forward primer with a 3′ terminal or penultimate nucleotide mismatch to the wild-type sequence. However, the selectivity of this technique is limited by inadvertent amplification of wild-type DNA, producing a false mutant template that is propagated by future PCR cycles. PCR restriction fragment length polymorphism mapping (PCR-RFLP) involves PCR amplification followed by digestion using a restriction enzyme that selectively cuts mutant DNA (Cohen et al. (2003) IOVS 44:7: 625-627). This approach is somewhat qualitative, and cannot be used to detect a low percentage of the mutant sequence. Wild-type-blocking PCR (WTB-PCR) utilizes the high affinity properties of locked nucleic acid (LNA)-substituted oligonucleotides to bind to the wild-type DNA sequence. The LNA blocker inhibits elongation of the primers by annealing to the mutation site on the wild-type sequence, thereby limiting wild-type amplification while permitting amplification of mutant DNA (Dominguez and Kolodney (2005) Oncogene 24: 6839-6834). However, elongation of the forward primer upstream of the mutation creates a partially extended product, which is more likely to amplify wild-type DNA due to its increased annealing temperature. To further increase the specificity of WTB-PCR, primer-blocking PCR (PB-PCR) was developed to avoid the generation of an extended forward primer. PB-PCR improves on the specificity of wild-type blocking PCR by moving the forward primer downstream, allowing the blocker to directly inhibit the binding of the primer.

Since the techniques described above can detect a few mutant cells in heterogeneous tissue, they may be applicable to clinical specimens containing as few as 1-10 melanoma cells circulating per mL of peripheral blood (Koyanagi et al. (2005) Clinical Chemistry 51:6: 981-988). Sensitive detection of these circulating melanoma cells may predict tumor progression (Pantel and Doeberitz (2000) Oncology 12: 95-101). However, these BRAF PCR-based techniques are not selective enough to detect less than 10 mutant copies in an excess of 10,000 wild-type copies, which is lower than the approximate 10⁶ fold ratio found in clinical samples from melanoma patients (Koyanagi et al. (2005) Clinical Chemistry 51:6: 981-988).

Current clinical approaches used to detect circulating melanoma cells rely on reverse-transcription PCR (RT-PCR). RT-PCR detects expression of melanocyte specific mRNAs such as tyrosinase. RT-PCR can sensitively detect as few as one melanoma cell per 10 mL of blood (Keilholz et al. (1998) Eur. J. Cancer 34: 750-753). However, since RT-PCR relies on mRNA expression, illegitimate transcription and poor reproducibility limit its clinical applicability (Id.). In order to avoid the inconsistency of RT-PCR assays, we developed primer-blocking allele-specific PCR (PBAS-PCR), a selective assay which directly targets BRAF-mutated genomic DNA in circulating melanoma cells. PBAS-PCR employs the additive effect of using an AS forward primer and an LNA blocker to maximize inhibition of wild-type amplification without adversely affecting detection of mutant sequences. In this example, we describe studies using PBAS-PCR to quantitatively assay defined cell mixtures and whole blood for the presence of mutated BRAF.

### Materials and Methods

Cell Lines, Tissue Sources, and Genomic DNA Preparation

HEK 293T cells, which do not contain oncogenic BRAF mutations, were purchased from Invitrogen Incorporated (Carlsbad, Calif., USA). A375M cells (ATCC catalog #CRL-1619), which are homozygous for the V599E BRAF mutation, were obtained as a gift from Dr. R. O. Hynes, M.I.T. (Cambridge, Mass.). Whole human blood was drawn from healthy volunteers with informed consent. Fresh blood was used for each experiment.

DNA and LNA Oligonucleotides

Sequence-specific oligonucleotides were designed and purchased from Integrated DNA Technologies (Coralville, Iowa, USA):

Non-allele-specific forward primer:

(SEQ ID NO: 4)

5′ AGG TGA TTT TGG TCT AGC TAC AG 3′.
Allele-specific forward primer:

(SEQ ID NO: 5)

5′ AGG TGA TTT TGG TCT AGC TAC AGA 3′.
Reverse primer:

(SEQ ID NO: 6)

5′ TAG TAA CTC AGC AGC ATC TCA GGG C 3′.
LNA blocker:

(SEQ ID NO: 7)

5′ GCT ACA GTG AGG G-3′.

*Bold-underline signifies LNA nucleotide

DNA Enrichment and Purification from Blood

A375M cells were counted and suspended to 10⁵ cells/mL 1×PBS by standard counting procedure. To obtain a concentration of 10 cells/mL blood, the cell suspension was serially diluted and added to the blood.

The RosetteSep CD45 Depletion Cocktail for Epithelial Tumor Cell Enrichment (product # 15122/15162) from StemCell Technologies (Canada) was used to isolate suspended tumor cells from whole blood. The RosetteSep crosslinks hematopoietic cells in whole blood to red blood cells, forming irnrununorosettes. When centrifuged over Ficoll-Paque ((catalog #07907) from Stem Cell Technologies), a buoyant density medium, the non-tumor cells and the free red blood cells pellet. Since tumor cells were not labeled with antibody, they settle at the plasma: Ficoll Paque interface. Modifications of the recommended procedure include: substitution of DMEM+2% FBS for PBS+2% FBS, and dilution of enriched cells with 8 mL 1×PBS 2% FBS.

Cells were pelleted out of PBS washing solution by centrifuging for 10 minutes at 2500 rpm. PBS washing solution was aspirated until 200 μL remained, resuspended, and transferred to a new microcentrifige tube. Samples were centrifuged at 10,000 rpm to remove PBS without disturbing the pellet. The sample then underwent whole genome amplification.

Primer-Blocking Allele-Specific PCR (PBAS-PCR)

Prior to PCR, WGA was performed using the REPLI-g Mini Kit (Qiagen Inc., Valencia, Calif., USA) to uniformly amplification genomic DNA with minimal sequence bias (Hosono et al. (2003) Genome Res. 13: 954). Following WGA, the samples were diluted 1:10 in TE for real-time PCR reactions. In preparation for a 25 μL real-time PCR reaction, a master mix was created with the following components: 12.5 μL QuantiTect SYBR Green Master Mix (Qiagen Incorporated, Valencia, Calif., USA); in substitution for the Titanium Taq PCR buffer, 1/1000 dilution of SYBR Green I probe, and mutated Taq polymerase (Stoffel fragment) used in previous assays), 7.5 μmol (0.75 μL) forward and reverse primer, 9 μL of nuclease-free H₂O, and 1 nmol (1 μL) LNA blocker. 1 μL of template DNA was added to this master mix. Mutant and wild-type samples were loaded into 96-well reaction plates and amplified in the ABI Prism 7000 Sequence Detection System (Applied Biosystems, North America) in a two-step procedure (PBAS-PCR). During the first step, samples were amplified for 30 cycles using non-allele-specific forward primers and LNA blocker. For the second step, the amplicon was diluted 1:500 in H₂O and was re-amplified in a 99 cycle reaction using an allele-specific forward primer and LNA blocker. Amplification parameters were as follows: Taq polymerase activation, 95° for 15 minutes; denaturation, 95° for 15 seconds; annealing, 66° for 30 seconds; and extension, 72° for 30 seconds. Post-amplification procedures included analysis of amplification plots and dissociation curves.

### Results

Combining AS Forward Primer and LNA Blocking to Increase Specificity.

Both AS primers and LNA blocking approaches have previously been shown to increase the selectivity of PCR-based mutation detection methods (Jarry et al 2004, Sadaat et al 2006 unpublished). We combined LNA blocking with AS primers in PBAS-PCR to maximize selectivity. The mechanism and molecular theory of PBAS-PCR is illustrated by FIG. 1. The AS forward primer selectively amplifies mutant BRAF DNA, while the LNA blocker directly inhibits the binding of the AS forward primer to the wild-type sequence without inhibiting amplification by binding to the mutant BRAF sequence.

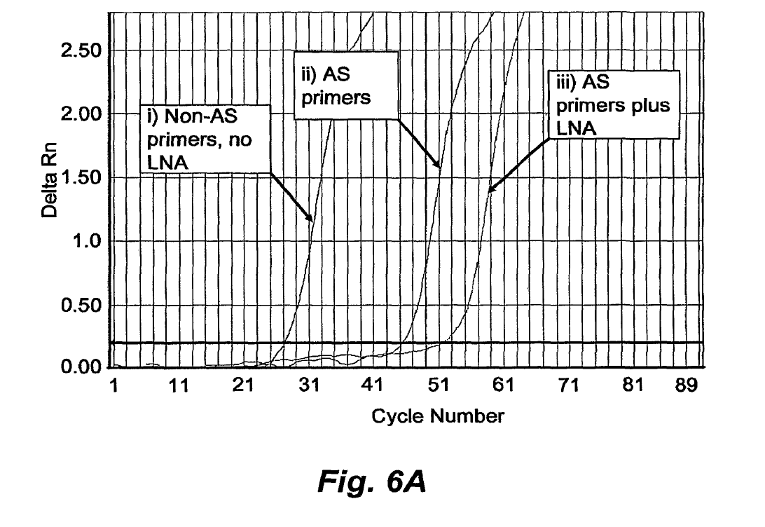

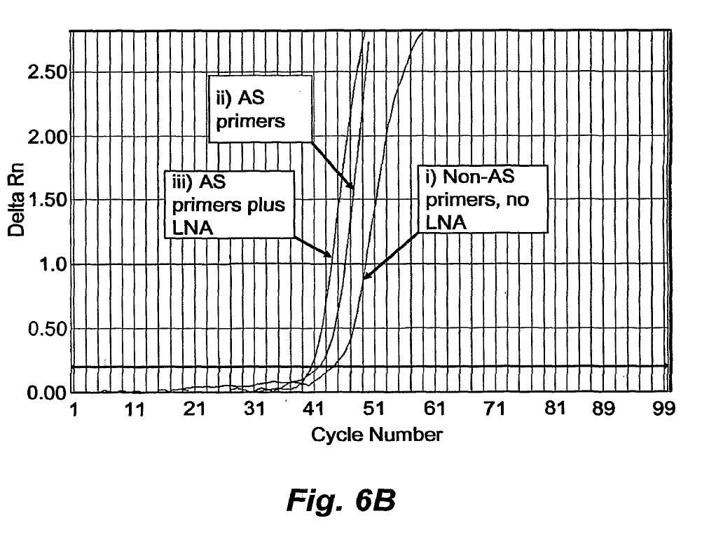

To demonstrate the additive effect of the AS forward primer and LNA blocker in the detection of mutated BRAF, we amplified genomic DNA isolated from wild-type (HEK 293T) and BRAF—mutated (A375M) cell lines. The substitution of an AS forward primer for a non-AS primer delayed wild-type amplification by 18 cycles (FIG. 56A). The addition of LNA blocker further increased the inhibition of wild-type amplification to 25 cycles. We determined the effects of the LNA blocker and AS forward primer by examining amplification plots of genomic mutant BRAF DNA (FIG. 6B). The AS forward primer amplified the mutant sequence five cycles earlier than the non-allele-specific primer, proving that the AS primer increases allelic discrimination between mutant and wild-type BRAF. The addition of an LNA blocker did not inhibit mutant amplification, demonstrating that the LNA blocker only targets wild-type BRAF. In summary, using both an AS forward primer and an LNA blocker in the same PCR reaction improves sensitivity of detection of mutated BRAF while inhibiting the amplification of wild-type sequences.

Detection of BRAF Mutations in Zenomic DNA Using Two-Step PBAS-PCR.

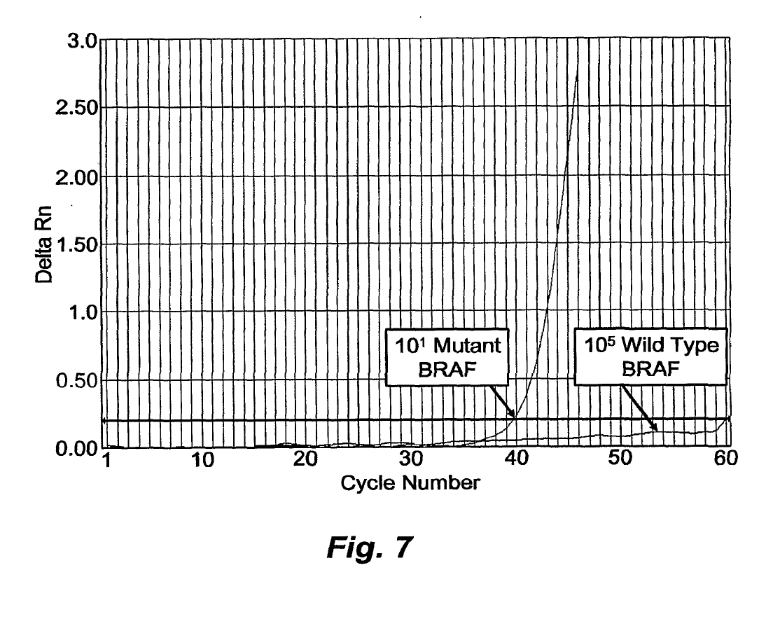

We developed two-step PBAS-PCR to exploit the additive effects of the AS forward primer and the LNA blocker. During both steps, LNA blocker inhibited wild-type amplification to decrease the wild-type:mutant ratio. In the first step (not shown), a non-AS forward primer was used so that polymerization errors would not generate an amplicon containing the BRAF mutation. In the second step, we used the 1:500 diluted amplicon from the first step with the AS forward primer to increase selectivity for mutant BRAF by only amplifying BRAF mutated DNA, As shown in FIG. 7, ten copies of mutant BRAF genomic DNA amplified at cycle 40, while amplification of 10⁵ copies of wild-type BRAF was undetectable even after 60 cycles. Since our assay creates a 20-cycle gap between the amplification of mutant and wild-type genomic samples, two-step PBAS-PCR can detect a low copy number of mutated BRAF in the presence of an excess of wild-type DNA.

### Detection of BRAF Mutated Melanoma Cells in Blood.

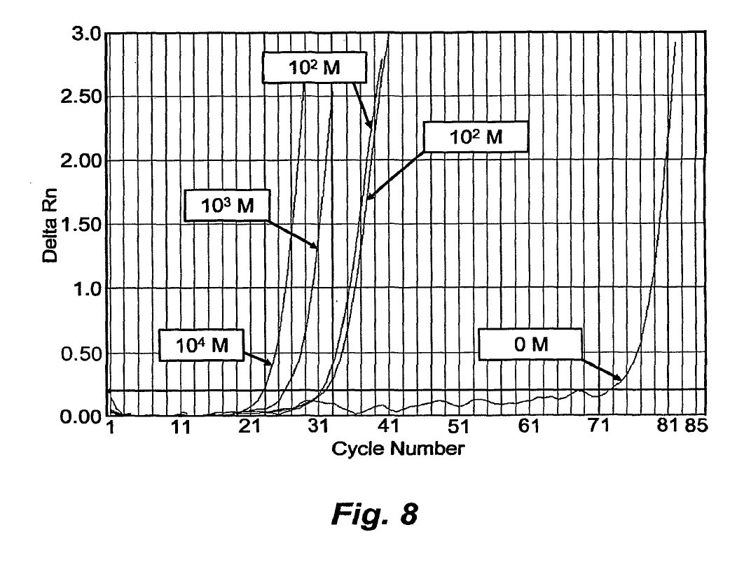

To determine if two-step PBAS-PCR could detect rare BRAF-mutated melanoma cells in peripheral blood, we spiked human whole blood with a defined number of A375M cells (range: 10¹ to 10⁴ cells/mL blood). We used a negative cell enrichment procedure to remove approximately 85% of the non-epithelial cells from blood by cross linking CD45-antibody-labeled hematopoietic cells to red blood cells (Lansdorp and Thomas (1990) Molecular Immunology 27: 659). Non-epithelial cells pellet after centrifugation, allowing a fraction enriched in epithelial cells, including melanoma cells, to be isolated. Whole genome amplification (WGA) was then performed on DNA from the enriched cells to increase the DNA copy number by approximately 4,000 fold, thereby facilitating earlier amplification of all samples (Hosono et al. (2003) Genome Res. 13: 954). FIG. 8 shows the results of a two-step PBAS-PCR procedure following epithelial cell enrichment and WGA. PBAS-PCR detected 10 melanoma cells after 20 cycles of amplification, while 73 cycles were necessary for detection of the amplicon from the epithelial enriched fraction of un-spiked blood. Amplification of mutant samples was also dose-dependent, indicating the reliability and accuracy of selective amplification by two-step PBAS-PCR.

### Discussion

Recent studies have shown that the detection of circulating melanoma cells in peripheral blood may prove useful in tracking tumor progression and predicting clinical outcomes (Ulmer et al. (2004) Clin. Cancer Res., 10: 531-537). Our study demonstrates that a novel allele-specific PCR approach combined with cell enrichment and WGA can detect circulating mutated cells in peripheral blood by amplifying rare BRAF mutated genomic DNA sequences. Our approach is a sensitive, reproducible assay that can detect as few as ten BRAF-mutated melanoma cells in one mL of blood. By using an antibody-mediated negative selection cell enrichment procedure in conjunction with WGA, we can remove the majority of non-melanoma cells while generating enough copies of mutant BRAF for PCR detection.

Dividing the PCR procedure into two steps maximized the selectivity of PBAS-PCR. The first step, in which non-allele-specific primers and LNA blocker were used, inhibits amplification of wild-type DNA. Since the non-AS forward primer does not contain the mutation site, priming errors were not propagated through synthesis of mutant template in the first step. During the second step, the additive effects of the AS-primer and the LNA blocker allow more selective amplification of mutant BRAF and further inhibition of wild-type amplification. PBAS-PCR selectively amplifies the mutant BRAF sequence in a dose dependent manner, allowing for relative quantification of the number of mutant cells present. Moreover, PBAS-PCR did not am plify wild-type samples until a much later cycle, thus facilitating clear identification of the BRAF mutation.

Detection of cancer specific mutations in genomic DNA may serve as an alternative to RT-PCR-based techniques which detect melanocyte-specific mRNA in circulating cancer. Recent studies suggest that RT-PCR can identify as few as one melanoma cell per 10 mL of blood by detecting tyrosinase expression (Koyanagi et al. (2005) Clinical Chemistry 51:6: 981-988). However, the inherent instability of mRNA introduces decrease the reproducibility of this approach. The resulting variability among different laboratories limits its clinical use. By using genomic DNA as a mutation marker, we avoid problems involving mRNA instability and false transcription of mRNA.

By quantifying and identifying genomic BRAF-mutated DNA, PBAS-PCR could potentially assist in early diagnosis and targeted melanoma treatment. For example, detection of BRAF-mutated melanoma cells in blood using PBAS-PCR could identify candidate patients who would benefit from agents targeting the constitutively activated MAPK signaling pathway since cells harboring oncogenic BRAF mutations are uniquely susceptible to these agents.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of preferentially amplifying a rare mutant nucleic acid in a population of nucleic acids comprising wild-type nucleic acids substantially in excess of said rare mutant nucleic acid, said method comprising:
carrying out a polymerase chain reaction (PCR) using a first primer and a second primer, where said first primer hybridizes with the region of said rare mutant nucleic acid comprising a mutation and said first primer and said second primer are not high affinity nucleic acids;
wherein the reaction mixture of said polymerase chain reaction also contains a high affinity nucleic acid analog, said high affinity nucleic acid analog being complementary to the region of a wild-type nucleic acid that is mutated in said mutant nucleic acid;
whereby binding of said high affinity nucleic acid analog to said wild-type nucleic acid prevents said first primer from binding to said wild-type nucleic acid thereby resulting in the preferential amplification of said rare mutant nucleic acid.

2. The method of claim 1, wherein said method further comprises:
comprises recovering the amplification product produced by said polymerase chain reaction;
diluting the amplification product;
carrying out said polymerase chain reaction again with said first primer, said second primer, and said high affinity nucleic acid analogue to further preferentially amplify said rare mutant nucleic acid.

3. The method of claim 1, wherein said rare mutant nucleic acid is present in said population at frequency of less than about 1 in 10³.

4-6. (canceled)

7. The method of claim 1, wherein said high affinity nucleic acid analogue is selected from the group consisting of a locked nucleic acid (LNA), a peptide nucleic acid (PNA), a hexitol nucleic acid (HNA), and a phosphoramidite.

8-10. (canceled)

11. The method of claim 1, wherein said first primer and said second primer independently range in length from about 12 nucleotides to about 60 nucleotides.

12-13. (canceled)

14. The method of claim 11, wherein said first primer is a forward primer.

15. The method of claim 1, wherein said high affinity nucleic acid analogue ranges in length from about 3 to about 25 bases.

16-17. (canceled)

18. The method of claim 1, wherein said high affinity nucleic acid analogue is present at a concentration of at least about 4-fold greater than the concentration of said first primer.

19. (canceled)

20. The method of claim 1, wherein said mutant nucleic acid comprises a plurality of point mutations.

21. A method of detecting a rare mutant nucleic acid in a population of nucleic acids comprising wild-type nucleic acids substantially in excess of said rare mutant nucleic acid, said method comprising:
hybridizing said rare mutant nucleic acid with a nucleic acid probe while blocking binding of said nucleic acid probe to the corresponding wild-type sequences by hybridizing said wild-type sequences to a high affinity nucleic acid analogue; and
detecting the hybridized nucleic acid probe or performing one or more PCR amplification reactions and detecting the amplification product comprising the mutant nucleic acid.

22. The method of claim 21, wherein said nucleic acid probe is labeled with a detectable label.

23. The method of claim 22, wherein said detectable label is selected from the group consisting of radioactive label, a radio-opaque label, an enzymatic label, a colorimetric label, and a fluorescent label.

24. The method of claim 21, wherein said rare mutant nucleic acid is present in said population at frequency of less than about 1 in 10³.

25. (canceled)

26. The method of claim 21, wherein said high affinity nucleic acid analogue is selected from the group consisting of a locked nucleic acid (LNA), a peptide nucleic acid (PNA), a hexitol nucleic acid (HNA), and a phosphoramidite.

27-29. (canceled)

30. The method of claim 21, wherein said nucleic acid probe ranges in length from about 12 nucleotides to about 100 nucleotides.

31-32. (canceled)

33. The method of claim 21, wherein said high affinity nucleic acid analogue ranges in length from about 3 to about 25 bases.

34-35. (canceled)

36. The method of claim 21, wherein said high affinity nucleic acid analogue is present at a concentration of at least about 4-fold greater than the concentration of said probe.

37. (canceled)

38. The method of claim 21, wherein said mutant nucleic acid comprises a plurality of point mutations.

39. A method of detecting a rare mutant nucleic acid in a population of nucleic acids comprising wild-type nucleic acids substantially in excess of said rare mutant nucleic acid, said method comprising:
carrying out a polymerase chain reaction (PCR) using a first primer and a second primer, where said first primer hybridizes with the region of said rare mutant nucleic acid comprising a mutation and said first primer and said second primer are not high affinity nucleic acids;
wherein the reaction mixture of said polymerase chain reaction also contains a high affinity nucleic acid analog, said high affinity nucleic acid analog being complementary to the region of a wild-type nucleic acid that is mutated in said mutant nucleic acid;
whereby binding of said high affinity nucleic acid analog to said wild-type nucleic acid prevents said first primer from binding to said wild-type nucleic acid thereby resulting in the preferential amplification of said rare mutant nucleic acid.

40. A method of performing a nucleic acid hybridization to a rare mutant nucleic acid in a population of nucleic acids comprising wild-type nucleic acids substantially in excess of said rare mutant nucleic acid, said method comprising:
hybridizing said rare mutant nucleic acid with a nucleic acid probe or primer, while blocking binding of said nucleic acid probe or primer to corresponding wild-type sequences by hybridizing said wild-type sequences to a high affinity nucleic acid analogue.

41. A method of detecting rare mutant nucleic acids in a complex population of nucleic acids, said method comprising:
contacting said population of nucleic acids with a high affinity nucleic acid that specifically hybridizes with the region of the wild-type sequence in which the mutant is expected to occur; thereby blocking the wild-type sequence; and
contacting the population of nucleic acids with a probe to detect the wild-type sequence; or
contacting the population of nucleic acids with a pair of PCR primers where one member of said pair hybridizes to a region of a nucleic acid in said population containing the mutation characterizing said rare mutants; and
amplifying the rare mutant nucleic acid.

42. A method of detecting a mutant nucleic acid in a mammal, said method compromising
providing a nucleic acid sample from said mammal;
hybridizing said mutant nucleic acid with a nucleic acid probe or PCR primer, while blocking binding of said nucleic acid probe or primer to corresponding wild-type sequences by hybridizing said wild-type sequences to a high affinity nucleic acid analogue; and
detecting the hybridized nucleic acid probe or performing one or more PCR amplification reactions and detecting the amplification product comprising the mutant nucleic acid.

43. A method of screening an agent for the ability to induce a mutation in a nucleic acid, said method comprising:
contacting a cell comprising said nucleic acid with said test agent;
providing a nucleic acid sample from said cell;
hybridizing said mutant nucleic acid with a nucleic acid probe or PCR primer, while blocking binding of said nucleic acid probe or primer to corresponding wild-type sequences by hybridizing said wild-type sequences to a high affinity nucleic acid analogue; and
detecting the hybridized nucleic acid probe or performing one or more PCR amplification reactions and detecting the amplification product comprising the mutant nucleic acid;
where the presence or increase in frequency of said mutation is an indicator that said test agent induces said mutation.

44. The method of claim 43, wherein said test agent is administered to or contacted to a non-human mammal comprising said cell.

45. The method of claim 43, wherein said test agent added to a cell culture comprising said cell.


SEQUENCE LISTING (7 sequences)

SEQ ID NO: 1 - DNA, 23 bp, Artificial
  PCR Primer
aggtgattttggtctagctacag

SEQ ID NO: 2 - DNA, 26 bp, Artificial
  PCR Primer
atcagtggaaaaatagcctcaattct

SEQ ID NO: 3 - DNA, 13 bp, Artificial
  Locked nucleic acid (LNA) blocker.
gctacagtgaggg

SEQ ID NO: 4 - DNA, 23 bp, Artificial
  PCR Primer
aggtgattttggtctagctacag

SEQ ID NO: 5 - DNA, 24 bp, Artificial
  PCR Primer
aggtgattttggtctagctacaga

SEQ ID NO: 6 - DNA, 25 bp, Artificial
  PCR Primer
tagtaactcagcagcatctcagggc

SEQ ID NO: 7 - DNA, 13 bp, Artificial
  PCR Primer
gctacagtgaggg